(19) United States
(12) Patent Application Publication
(10) Pub. No.: US 2024/0366227 A1
(43) Pub. Date: Nov. 7, 2024

(54) DEVICES FOR TREATMENT OF VASCULAR DEFECTS

(71) Applicant: MICROVENTION, INC., Aliso Viejo, CA (US)

(72) Inventors: Hung P. Tran, Midway City, CA (US); Todd Hewitt, Laguna Niguel, CA (US); Parker Milhous, Santa Ana, CA (US); Karishma Desai, Irvine, CA (US)

(21) Appl. No.: 18/648,963

(22) Filed: Apr. 29, 2024

Related U.S. Application Data
(63) Continuation of application No. PCT/US22/48938, filed on Nov. 4, 2022.
(60) Provisional application No. 63/276,537, filed on Nov. 5, 2021.

(51) Int. Cl. A61B 17/12; A61B 17/00

(52) U.S. Cl. CPC A61B 17/12113; A61B 17/12036; A61B 17/12172; A61B 2017/00477; A61B 2017/1205

(57) ABSTRACT

Devices and methods for treatment of a patient's vasculature are described. Embodiments may include a permeable implant made from a plurality of elongate filaments that are woven together. The implant may have a first unrestrained preset configuration comprising a dome portion and a brim portion, having a distal end with an outer surface having a convex shape. The implant may have a second deployed configuration that is an inversion of the first unrestrained configuration. The second deployed configuration has an open distal end and has an inner surface having a concave shape.

### CROSS-REFERENCE TO RELATED APPLICATIONS

This application is a continuation of International Application No. PCT/US22/48938, filed Nov. 4, 2022, which claims the benefit of U.S. Provisional Application Ser. No. 63/276,537, filed Nov. 5, 2021, both of which are hereby expressly incorporated by reference in their entireties for all purposes.

### STATEMENT REGARDING FEDERALLY SPONSORED RESEARCH OR DEVELOPMENT

Not applicable.

### FIELD OF THE INVENTION

Embodiments of devices and methods herein are directed to blocking a flow of fluid into a small interior chamber of a saccular cavity or vascular defect within a mammalian body. More specifically, embodiments herein are directed to devices and methods for treatment of a vascular defect of a patient including some embodiments directed specifically to the treatment of cerebral aneurysms of patients.

### BACKGROUND OF THE INVENTION

The mammalian circulatory system is comprised of a heart, which acts as a pump, and a system of blood vessels which transport the blood to various points in the body. Due to the force exerted by the flowing blood on the blood vessel the blood vessels may develop a variety of vascular defects. One common vascular defect known as an aneurysm results from the abnormal widening of the blood vessel. Typically, vascular aneurysms are formed as a result of the weakening of the wall of a blood vessel and subsequent ballooning and expansion of the vessel wall. If, for example, an aneurysm is present within an artery of the brain, and the aneurysm should burst with resulting cranial hemorrhaging, death could occur.

Surgical techniques for the treatment of cerebral aneurysms typically involve a craniotomy requiring creation of an opening in the skull of the patient through which the surgeon can insert instruments to operate directly on the patient's brain. For some surgical approaches, the brain must be retracted to expose the parent blood vessel from which the aneurysm arises. Once access to the aneurysm is gained, the surgeon places a clip across the neck of the aneurysm thereby preventing arterial blood from entering the aneurysm. Upon correct placement of the clip the aneurysm will be obliterated in a matter of minutes. Surgical techniques may be effective treatment for many aneurysms. Unfortunately, surgical techniques for treating these types of conditions include major invasive surgical procedures which often require extended periods of time under anesthesia involving high risk to the patient. Such procedures thus require that the patient be in generally good physical condition in order to be a candidate for such procedures.

Various alternative and less invasive procedures have been used to treat cerebral aneurysms without resorting to major surgery. One approach to treating aneurysms without the need for invasive surgery involves the placement of sleeves or stents into the vessel and across the region where the aneurysm occurs. Such flow diverter devices maintain blood flow through the vessel while reducing blood pressure applied to the interior of the aneurysm. Certain types of stents are expanded to the proper size by inflating a balloon catheter, referred to as balloon expandable stents, while other stents are designed to elastically expand in a self-expanding manner. Some stents are covered typically with a sleeve of polymeric material called a graft to form a stent-graft. Stents and stent-grafts are generally delivered to a preselected position adjacent a vascular defect through a delivery catheter. In the treatment of cerebral aneurysms, covered stents or stent-grafts have seen very limited use due to the likelihood of inadvertent occlusion of small perforator vessels that may be near the vascular defect being treated.

In addition, current uncovered stents are generally not sufficient as a stand-alone treatment. In order for stents to fit through the microcatheters used in small cerebral blood vessels, their density is usually reduced such that when expanded there is only a small amount of stent structure bridging the aneurysm neck. Thus, they do not block enough flow to cause clotting of the blood in the aneurysm and are thus generally used in combination with vaso-occlusive devices, such as the coils discussed above, to achieve aneurysm occlusion.

Some procedures involve the delivery of embolic or filling materials into an aneurysm. The delivery of such vaso-occlusion devices or materials may be used to promote hemostasis or fill an aneurysm cavity entirely. Vaso-occlusion devices may be placed within the vasculature of the human body, typically via a catheter, either to block the flow of blood through a vessel with an aneurysm through the formation of an embolus or to form such an embolus within an aneurysm stemming from the vessel. A variety of implantable, coil-type vaso-occlusion devices are known. The coils of such devices may themselves be formed into a secondary coil shape, or any of a variety of more complex secondary shapes. Vaso-occlusive coils are commonly used to treat cerebral aneurysms but suffer from several limitations including poor packing density, compaction due to hydrodynamic pressure from blood flow, poor stability in wide-necked aneurysms, and complexity and difficulty in the deployment thereof as most aneurysm treatments with this approach require the deployment of multiple coils. Coiling is less effective at treating certain physiological conditions, such as wide neck cavities (e.g. wide neck aneurysms) because there is a greater risk of the coils migrating out of the treatment site.

A number of aneurysm neck bridging devices with defect spanning portions or regions have been attempted, however, none of these devices have had a significant measure of clinical success or usage. A major limitation in their adoption and clinical usefulness is the inability to position the defect spanning portion to assure coverage of the neck. Existing stent delivery systems that are neurovascular compatible (i.e. deliverable through a microcatheter and highly flexible) do not have the necessary rotational positioning capability. Another limitation of many aneurysm bridging devices described in the prior art is the poor flexibility. Cerebral blood vessels are tortuous, and a high degree of flexibility is required for effective delivery to most aneurysm locations in the brain.

What has been needed are devices and methods for delivery and use in small and tortuous blood vessels that can substantially block the flow of blood into an aneurysm, such as a cerebral aneurysm, with a decreased risk of inadvertent aneurysm rupture or blood vessel wall damage. In addition, what has been needed are methods and devices suitable for blocking blood flow in cerebral aneurysms over an extended period of time without a significant risk of deformation, compaction, or dislocation.

Intrasaccular occlusive devices are part of a newer type of occlusion device used to treat various intravascular conditions including aneurysms. They are often more effective at treating these wide neck conditions, or larger treatment areas. The intrasaccular devices comprise a structure that sits within the aneurysm and provides an occlusive effect at the neck of the aneurysm to help limit blood flow into the aneurysm. The rest of the device comprises a relatively conformable structure that sits within the aneurysm helping to occlude all or a portion of the aneurysm. Intrasaccular devices typically conform to the shape of the treatment site. These devices also occlude the cross section of the neck of the treatment site/aneurysm, thereby promoting clotting and causing thrombosis and closing of the aneurysm over time. In larger aneurysms, there is a risk of compaction where the intrasaccular device can migrate into the aneurysm and leave the neck region.

For any sized aneurysm, there may be numerous different types of sizes of occlusive devices that could be chosen by the physician to treat the aneurysm, where the devices may differ in height and diameter. The implants may also have different expanded shapes, e.g., barrel or spherical shape. Thus, many different sized and models of implants may have approximately the same volume as the aneurysm to be treated, and therefore are an acceptable “volume match” for the aneurysm.

Alternatively, the implant may be less than the total height of the aneurysm. Some devices are designed to only fill approximately half of the aneurysm. These implants may be able to be used in different sized aneurysms because volume matching is not required. These implants, however, may not optimally cover the necks of wider aneurysms. For example, devices having a tapered proximal end may not suitably cover the neck of wide neck aneurysms. Moreover, an ill-fitting device may move distally into the aneurysm and not stay anchored at the neck.

There is a need for occlusive devices that have a size that is suitable to treat multiple different sized aneurysms with varying neck size.

The following embodiments address this issue by utilizing a device having a preset expanded shape that can conform to fit into and treat numerous sizes of aneurysms, including wide-neck aneurysms.

### SUMMARY OF THE INVENTION

An occlusion device is described that is used to treat a variety of conditions, including aneurysms and neurovascular aneurysms. In some embodiments, the occlusion device is configured as an intrasaccular device.

### BRIEF DESCRIPTION OF THE DRAWINGS

These and other aspects, features, and advantages of which embodiments of the invention are capable of will be apparent and elucidated from the following description of embodiments of the present invention, reference being made to the accompanying drawings, in which:

The various figures included show the occlusive device according to one or more embodiments.

FIG. 1 shows a device for treatment of an aneurysm.

FIGS. 2A and 2B show a diagram of an exemplary device and the exemplary device for treatment of an aneurysm having an unconstrained configuration having an inverted shape with a closed distal end and an open proximal end.

FIG. 3 shows the exemplary device in a delivery catheter.

FIGS. 4A and 4B show a diagram of the exemplary device and the exemplary device of FIGS. 2A and 2B deployed in the aneurysm, the exemplary device having an open distal end and a closed proximal end.

FIGS. 5A-5E depict the exemplary device being deployed in an aneurysm.

FIG. 6 depicts a tubular mesh having an opening defined by loops of the filament.

### DESCRIPTION OF EMBODIMENTS

The presented embodiments shall generally relate to occlusive devices that can be used to treat different sized aneurysms.

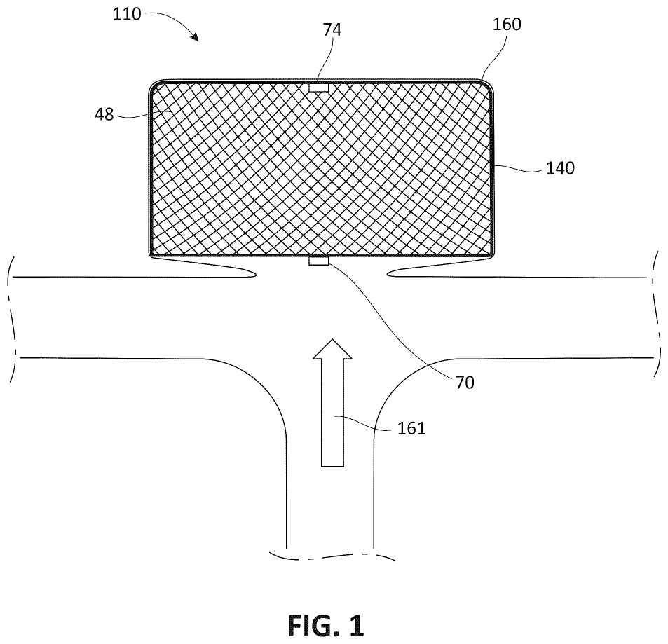

One method of choosing a device for placement into a particular aneurysm is to match the volume of the device 110 with the volume of the aneurysm. As seen in FIG. 1, for devices 110 that have a permeable shell 140 having an approximate barrel or spherical shape, the physician may choose an implant according to a “+1/−1” strategy. For example, the permeable shell 140 of the device 110 chosen may have a diameter of about 1 mm greater than a mean diameter of the aneurysm and a height of about 1 mm less that a minimum height of the aneurysm. Oversizing the permeable shell 140 by about 1 mm relative to the aneurysm diameter may ensure that there is a slight compression force on the implant after it is deployed in the aneurysm. This compression force may help the permeable shell 140 to maintain a stable position after implantation as blood flows into and out of the aneurysm. When the permeable shell 140 compresses in diameter by about 1 mm to fit within the aneurysm, the permeable shell 140 may elongate by about 1 mm in height, thereby occupying approximately the full aneurysm volume. Thus, the permeable shell 140 selected may be approximately equal to the aneurysm volumetrically but initially larger in diameter. This diametric oversizing may provide friction against the aneurysm wall after deployment, and “volume matching” may allow the permeable shell 140 to fill the aneurysm fully, thereby maintaining a stable position as hemodynamic and clot contraction forces (represented by the arrow 161) exert pressure on the implant over time.

For example, when treating an aneurysm with a mean diameter of about 5 mm diameter, the physician may choose between a device 110 having a diameter×height of 6×3, 6×4, 6×5 (e.g., barrel shape), or a spherical device having a diameter of about 6 mm. Similarly, an aneurysm with a mean diameter of about 6 mm can be treated with a device 110 having a diameter×height of 7×3, 7×4, or 7×5 (e.g., barrel shape), or a spherical device having a diameter of about 7 mm.

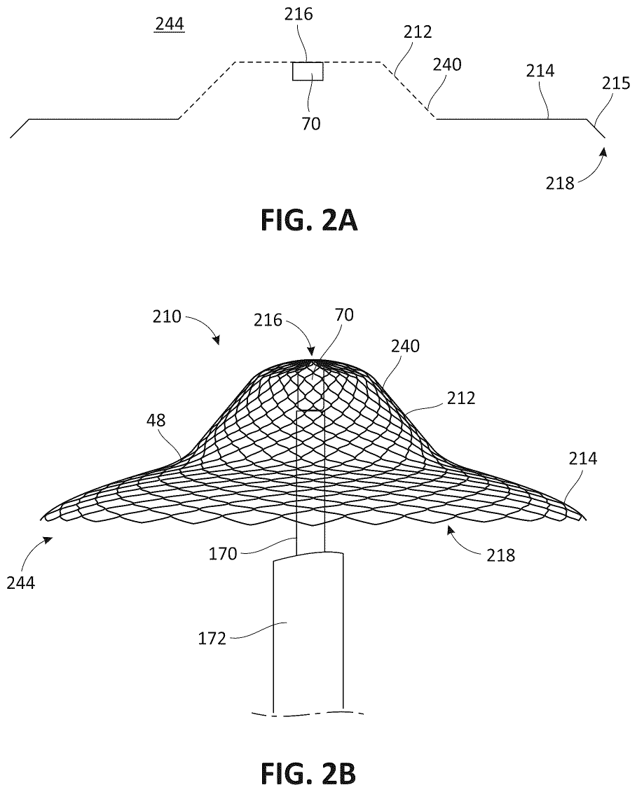

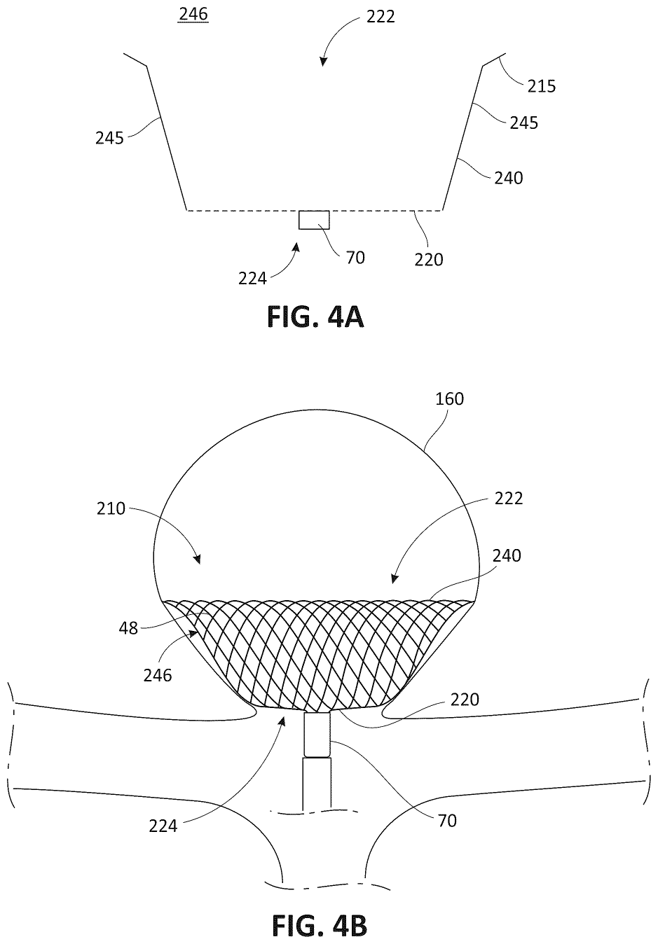

In alternative embodiments, the device may have an unrestrained, expanded, preset, heat-set, “free air,” or unconstrained shape and a different expanded or deployed shape when it is deployed in the aneurysm, where it is constrained by the aneurysm walls. The expanded preset shape may be capable of being deformed by compressive forces of the aneurysm wall into the different expanded shape. As seen in FIG. 2, the permeable shell 240 may have an unconstrained, expanded, preset, or heat-set configuration 244, as it exists in “free air,” that is umbrella- or hat-shaped. In some embodiments, the expanded preset shape may be heat set in the umbrella or hat shape and may have a recess near the marker band 70. When deployed, as seen in FIGS. 4A-4B, the recess may result in a flatter bottom or proximal surface that better conforms to wide-neck geometries. The recess geometry may be optimized to achieve optimal deployment and acute performance characteristics. In the deployed configuration, the permeable shell may no longer have a recess geometry at the proximal end, such that the marker band may not be recessed relative to a plane defined by a proximal-most point of the permeable shell. In other embodiments, the expanded preset shape may be heat set in the umbrella or hat shape and may not have a recess near the marker band 70.

As seen in FIGS. 2A-2B, the expanded, preset, heat-set, or unconstrained configuration of the implant may resemble an umbrella or a hat in “free air”. Each of the plurality of filaments that form the device may have a first end and a second end. In some embodiments, both of the first and second ends of each of the plurality of filaments may be gathered in a single hub or marker band 70. In other embodiments, only a first end of each of the plurality of filaments may be gathered in a hub or marker band 70. The plurality of filaments may be braided or looped to form a basket or cage. In some embodiments, a middle portion of each of the plurality of filaments may form a loop at the proximal end 218 of the unrestrained configuration 244.

The expanded, preset configuration 244 may include a dome-shaped portion 212 that connects to a brim portion 214 in “free air.” The brim portion 214 may also curve downward or have a slightly convex or sloping shape. In some embodiments, the brim portion 214 may not be substantially flat, straight, or horizontal. The brim portion 214 may also optionally have a lip 215 that slopes downward from the brim portion at a different angle that the downward curve of the brim portion 214. The lip portion 215 may have a larger slope than the brim portion 214. The expanded, preset unconstrained configuration 244 may also be an inverted configuration that has an outer and inner surface. The hub or marker band 70 may be located in an inner cavity formed by an inner surface of the dome-shaped portion 212 such that a distal end 216 of the unrestrained, expanded, heat-set, or pre-set configuration 244 is inverted.

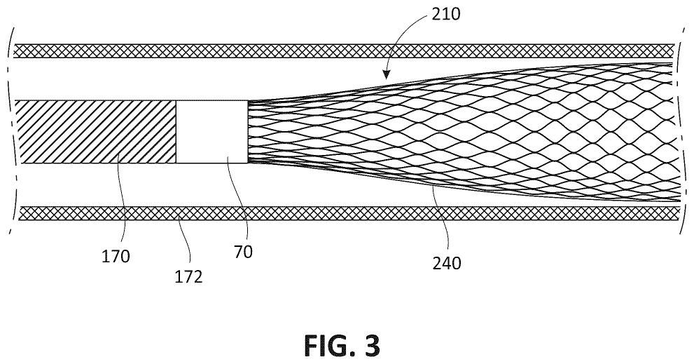

As seen in FIG. 3, the permeable shell 240 of the device 210 may have a radially constrained elongated configuration for delivery within a microcatheter. The hub or marker band 70 may be detachably coupled to a pusher 170. In the elongated configuration, the permeable shell may be extended in a distal direction from the hub or marker band 70.

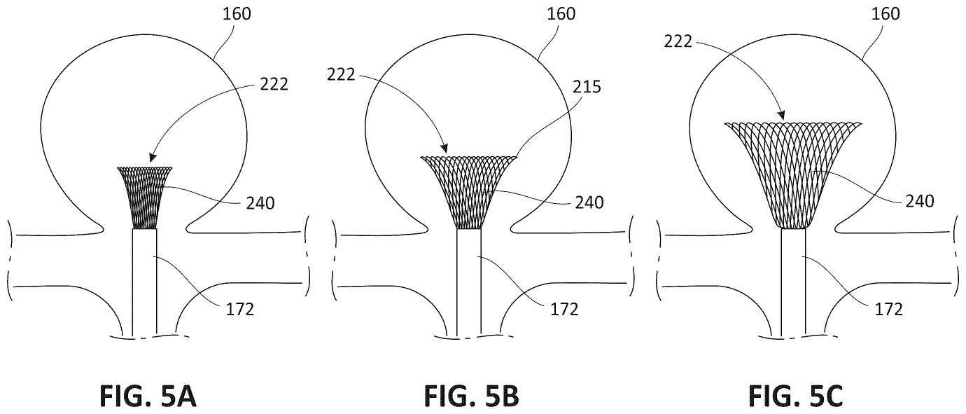

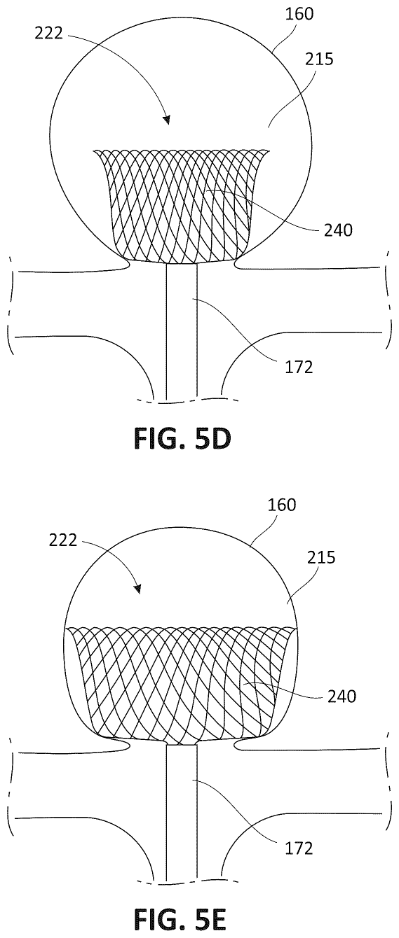

As seen in FIGS. 4A-4B and 5A-5E, the permeable shell 240 may assume a different (other than the hat-shaped or umbrella unrestrained, preset, “free air” configuration) expanded shape 246 when it is deployed in an aneurysm 160. The permeable shell 240 may be in an inverted configuration in the microcatheter 172, as seen in FIG. 3, with the permeable shell 240 extending distally from the hub or marker band 70, where the permeable shell has an open distal end 222. The permeable shell 240 may substantially conform to the shape of the aneurysm. Because the diameter of the distal end of the permeable shell may be larger than a maximum diameter of the aneurysm (e.g., about or at least about 1 mm larger), the sides of the permeable shell 240 may be compressed, causing the permeable shell to lengthen to a height greater than the height of the unrestrained expanded preset configuration 244. The lip 215 of the permeable shell 240 may be outwardly extend at an angle similar to a tulip, bowl, cup, or teacup shape, which may increase the friction and aid in keeping the permeable shell 240 positioned in the inner cavity of the aneurysm 160. The substantially flat portion 220 may occupy the opening of the aneurysm 160 at the neck. The substantially flat portion 220 of the deployed shape may correspond to the sides and top of the dome-shaped portion 212 seen in FIG. 2A. A proximal portion of the permeable shell in the deployed configuration may have an inverted frustoconical shape.

In one embodiment, in order to deploy the implant, a microcatheter 172 may be directed adjacent to a neck of the aneurysm 160. As seen in FIGS. 5A-5E, a pusher 170 may advance the permeable shell 240 out of the microcatheter 172 and into an inner cavity of the aneurysm 160, where the permeable shell 240 has an open distal end 222 with a lip 215. Because the permeable shell 240 is biased towards its preset, heat-set, “free air” configuration, as the permeable shell is further advanced out of the distal opening of the microcatheter 172, the open distal end 222 continues to expand. As the permeable shell is further advanced out of the distal opening of the microcatheter 172, at least a portion of the side walls of the permeable shell 240 contact the walls of the inner cavity of the aneurysm 160, which may prevent the permeable shell 240 from assuming the preset, heat-set, “free air” configuration. Instead, the permeable shell 240 may conform to the walls of the aneurysm 160 such that the expanded deployed state has an open distal end 222 and resembles a bowl or a tulip. The dome-shaped portion 212 of the heat-set configuration may become the substantially flat portion positioned over the neck of the aneurysm 160. Because the permeable shell 240 is biased towards its preset, heat-set configuration, the sidewalls of the permeable shell (formerly the brim portion 212 lip portion 215) may exert a pressure on the aneurysm wall, which may assist in keeping the permeable shell 240 properly positioned in the aneurysm 160.

In another embodiment, in order to deploy the implant, a microcatheter 172 may be directed adjacent to a neck of the aneurysm 160. A pusher 170 may advance the permeable shell 210 out of the microcatheter 172 and into an inner cavity of the aneurysm 160, where the permeable shell 240 may assume a partially inverted configuration having an open proximal end, where the hub 70 is sitting in an inner cavity of the dome-shaped portion 212, where the brim portion 214 may be constrained by the aneurysm walls. As the pusher 170 is withdrawn from the inner cavity, the permeable shell may invert such that the hub 70 is no longer sitting within the inner cavity of the dome-shaped portion 210. As the pusher 170, which is attached to the hub 70, is withdrawn proximally, the dome-shaped portion 212 may invert and assume the substantially flat portion 220 seen in FIGS. 4A-4B, while the brim-shape portion 214 may conform to the walls of the aneurysm such that the expanded deployed state has an open distal end 222 and resembles a bowl or a tulip.

The deployed shape of the implant may be an inversion of the unrestrained, “free air” shape of the implant. As seen in FIGS. 4A-4B, after deployment in the aneurysm 160, the device 244 may have a bowl shape or tulip shape having a substantially flat portion 220 at the proximal end 224 and an open distal end 222. In the restrained expanded state within the vascular defect (e.g., aneurysm), the proximal end 214 of the unrestrained device (see, e.g., FIG. 2B) may become the distal end of the restrained expanded configuration 246 of the device deployed in the aneurysm. In the restrained, or deployed, configuration 246, in some embodiments, the loops made from a middle portion of each of the plurality of filaments and may form the edge of the open distal end 222.

As seen in FIGS. 2A and 4A, the dome-shaped portion 212 may form the substantially flat portion 220, and the brim portion 214 may form the sides 245 of the expanded device. A length of the dome-shaped portion 212 and/or the substantially flat portion may be between about 2.5 inches to about 4.5 inches, alternatively between about 2.8 inches to about 4.2 inches, alternatively between about 3.0 inches to about 4.0 inches, alternatively between about 3.2 inches to about 3.8 inches.

A length of the brim portion 214 and/or the brim portion 214 and lip portion 215 combined may equal or substantially equal a height of the restrained expanded configuration 246. A length of the brim portion 214 and/or the brim portion 214 and lip portion 215 combined of the preset, “free air” configuration, which may correspond to a height of the expanded configuration, may be between about 1.0 to about 3.0 inches, about 1.5 to about 3.0 inches, alternatively between about 1.5 to about 2.5 inches, alternatively between about 1.5 to about 2.25 inches, about 1.0 to about 2.0 inches. The length of the brim portion 214 and/or the brim portion 214 and lip portion 215 combined of the preset, “free air” configuration, which may correspond to a height of the expanded configuration, may be greater than about 20%, alternatively greater than about 30%, alternatively greater than about 40%, alternatively greater than about 50% than the length of the substantially flat portion. The length of the brim portion 214 and/or the brim portion 214 and lip portion 215 combined of the preset, “free air” configuration, which may correspond to a height of the expanded configuration, which may correspond to a height of the expanded configuration, may be between about 20% and about 90%, alternatively between about 30% and about 90%, alternatively between about 40% and about 90%, alternatively between about 50% and about 90% of the length of the substantially flat portion. In some embodiments, the length of the sides adjacent the aneurysm sidewalls may not change because they are defined by (1) the edge of the brim portion (or if the lip is present, the edge of the lip portion), and (2) the inflection point between the dome portion and brim portion.

Because the permeable shell 240 is heat set into a different shape that is inverted with reference to the expanded, deployed shape in the aneurysm, the permeable shell 240 may exert a pressure against the walls of aneurysm as it is biased to assume its preset inverted (or “free air”) configuration. The pressure may assist in keeping the permeable shell 240 in a proper position within the aneurysm 160. One advantage of the bowl shape of device 210 is that the physician may consider fewer models in determining an appropriate size for the aneurysm.

The permeable shell 240 of the device 210 may have a radially constrained elongated configuration for delivery within a microcatheter. The permeable shell 240 may have an expanded preset configuration 244 or unconstrained configuration with a hat-shaped longitudinally shortened configuration relative to the radially constrained state. Once deployed into an aneurysm 160, however, as seen in FIGS. 4A-4B, the permeable shell may assume a different (other than the hat-shaped preset configuration) expanded shape 246 in response to the proximal withdrawal of the pusher and the compressive forces from the aneurysm walls. The permeable shell 240 may substantially conform to the shape of the aneurysm. Because the diameter of the distal end of the permeable shell may be larger than a maximum diameter of the aneurysm (e.g., about or at least about 1 mm larger), the sides of the permeable shell 240 may be compressed.

In the expanded deployed configuration, in some embodiments, the hub may not be recessed relative the mesh at the proximal end of the permeable shell. In some embodiments, the expanded deployed configuration may not have a concave portion at the proximal end formed by the outer surface of the permeable shell.

The permeable shell 240 of the implant may be made from a braided tubular mesh. Mechanisms and methods for forming the tubular braided meshes are described in more detail in U.S. Pat. Nos. 8,261,648, 8,826,791, and US 2021/0275184, which are hereby expressly incorporated by reference in their entireties for all purposes. The mesh or braided portion may be made from a plurality of filaments in a woven structure. The plurality of filaments that make up the mesh or braided portion may be made from nitinol, stainless steel, drawn filled tubes (e.g., platinum or tantalum core with a nitinol jacket), platinum, platinum alloys such as platinum/tungsten, super-elastic metals such as NiTiNibY, high strength metals such as CoCr, or a mixture thereof.

Some device embodiments may be formed using about 10 filaments to about 300 filaments, alternatively about 10 filaments to about 100 filaments, alternatively about 60 filaments to about 80 filaments, alternatively about 72 to about 216 filaments, alternatively about 150 to about 300 filaments. Some embodiments of a permeable shell may include about 70 filaments to about 300 filaments, alternatively, about 100 filaments to about 200 filaments. The wires or filaments may have a diameter or a transverse dimension of about 0.0005 inches to about 0.003 inches, alternatively about 0.001 inches to about 0.003 inches, alternatively about 0.0015 inches to about 0.0025 inches, alternatively about 0.0008 inches to about 0.004 inches. The elongate resilient filaments in some cases may have an outer transverse dimension or diameter of about 0.0005 inches to about 0.005 inches, alternatively about 0.001 inches to about 0.003 inches, alternatively about 0.0004 inches to about 0.002 inches.

In some embodiments, the mesh may be made from a mixture of filaments of different types of material (e.g., nitinol and DFT) and/or different sizes of filaments. In some embodiments, the mesh may be made from a non-uniform, or a relatively non-uniform distribution of filaments. In other embodiments, the mesh may be made from a relatively uniform distribution of filaments.

The wires or filaments making up a single mesh may have different transverse diameters and may also be made of different materials. For some device embodiments that include filaments of different sizes, the large filaments of the permeable shell may have a transverse dimension or diameter that is about 0.001 inches to about 0.004 inches and the small filaments may have a transverse dimension or diameter of about 0.0004 inches to about 0.0015 inches, or alternatively, about 0.0004 inches to about 0.001 inches. In addition, a difference in transverse dimension or diameter between the small filaments and the large filaments may be less than about 0.004 inches, alternatively, less than about 0.0035 inches, alternatively, less than about 0.002 inches. For embodiments of permeable shells that include filaments of different sizes, the number of small filaments of the permeable shell relative to the number of large filaments of the permeable shell may be about 2 to 1 to about 15 to 1, more specifically, about 2 to 1 to about 12 to 1, and even more specifically, about 4 to 1 to about 8 to 1.

Suitable materials and sizes of wires for constructing mesh implants are described in US 2017/0095254, US 2016/0249934, US 2016/0367260, US 2016/0249937, and US 2018/0000489, all of which are hereby expressly incorporated by reference in their entirety for all purposes.

In some embodiments, the implant may have a permeable shell having multiple layers of mesh that is made from a single braided mesh that is folded over, with only a proximal hub or marker band at the proximal near the aneurysm neck. The permeable shell may include two layers where the mesh is folded over once, alternatively three layers where the mesh is folded twice, alternatively four layers where the mesh is folded three times. Methods for making multiple layer permeable shells are described in more detail in US 2022/0257260, which is hereby expressly incorporated by reference in its entirety for all purposes. In some embodiments, the implant may be a double layer and may be made by folding over the mesh such that both ends of the filaments are at a single end of the device. A proximal hub or marker band 70 may hold both ends of each filament of the plurality of filaments.

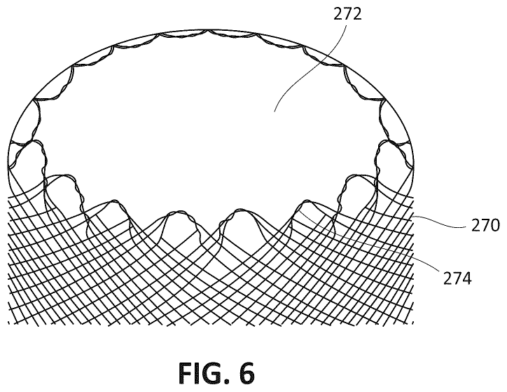

In some embodiments, the implant may have a permeable shell having only a single layer of mesh. The proximal hub may only hold one end (e.g., the proximal end) of each of the filaments of the plurality of filaments making up the mesh and the other end (e.g., the distal end) of each of the filaments of the plurality of filaments may be free and unbound at the open distal end 222. Alternatively, the other ends of the filaments may be woven into the mesh of the permeable shell 240. Alternatively, the second ends of the filaments may be gathered and held together by an additional hub. The additional hub may sit in the open cavity formed by the deployed permeable shell and sit towards the proximal end 224 of the device 210. The additional hub may sit close to or adjacent the proximal hub, separated by the mesh layer or layers making up the permeable shell 240. Alternatively, the implant may be a single layer and may be made from a single braided mesh that may be constructed on a castellated mandrel, as described in US 2021/0275184, which was previously expressly incorporated by reference in its entirety for all purposes. The braided mesh 270 may be constructed using the castellated mandrel such that filaments may be formed into loops 274 and an opening 272 may be centrally located at the distal end, as seen in FIG. 6. The ends of the plurality of filaments may be secured relative to each other at the proximal end, e.g., in proximal hub or marker band 70.

All features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

As used herein and in the appended claims, the singular forms “a,” “an,” and “the” include plural referents unless the context clearly dictates otherwise.

Aspects of the invention are set out in the independent claims and preferred features are set out in the dependent claims. The preferred features of the dependent claims may be provided in combination in a single embodiment and preferred features of one aspect may be provided in conjunction with other aspects.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

In many embodiments, a device for treatment of a patient's cerebral aneurysm includes a permeable shell made from a plurality of elongate filaments, wherein each of the plurality of filaments has a first end and a second end, wherein the first end of each of the plurality of elongate filaments are gathered in a hub, wherein the permeable shell has a first unrestrained preset configuration comprising a dome portion and a brim portion, wherein the dome portion comprises an outer surface, an inner surface, and an inner cavity defined by the inner surface, and wherein the hub is located in the inner cavity in the first unrestrained preset configuration, and wherein the permeable shell is configured to assume a second restrained configuration when deployed in the patient's cerebral aneurysm, wherein the second restrained configuration comprises an open distal end.

In some embodiments, the first unrestrained preset configuration has a hat shape. In some embodiments, the hat shape further comprises a lip portion. In some embodiments, the lip portion extends at an acute angle from the brim portion.

In some embodiments, the first unrestrained preset configuration has an umbrella shape.

In some embodiments, the hub is located at a distal end of the first unrestrained preset configuration.

In some embodiments, the second restrained configuration has a bowl shape.

In some embodiments, the second constrained configuration further comprises a substantially flat portion at a proximal end.

In some embodiments, the second restrained configuration has a tulip shape.

In some embodiments, the second restrained configuration has a cup shape.

In some embodiments, the hub is located at a proximal end of the second restrained configuration.

In some embodiments, the first end and the second end of each of the plurality of elongate filaments are gathered in a hub.

In some embodiments, each of the plurality of elongate filaments comprises a middle portion, and wherein the middle portion forms a loop at a distal end of the second restrained configuration.

In some embodiments, each of the plurality of elongate filaments comprises a middle portion, and wherein the middle portion forms a loop at a proximal end of the first unrestrained preset configuration.

In some embodiments, a distal end of the permeable shell in the first unrestrained preset configuration is inverted.

In some embodiments, a distal end of the permeable shell in the first unrestrained preset configuration has an outer convex surface.

In some embodiments, a distal end of the permeable shell in the second restrained configuration has an inner concave surface.

In some embodiments, the plurality of elongate filaments are arranged in a braided mesh. In some embodiments, the permeable shell comprises a single layer of the braided mesh. In some embodiments, the permeable shell comprises a double layer of the braided mesh. In some embodiments, the permeable shell comprises multiple layers of the braided mesh.

In many embodiments, a method for treating a cerebral aneurysm having an interior cavity and a neck includes the steps of: advancing an implant in a microcatheter to a region of interest in a cerebral artery, wherein the implant comprises a permeable shell made from a plurality of elongate filaments, wherein each of the plurality of filaments has a first end and a second end, wherein the first end of each of the plurality of elongate filaments are gathered in a hub, wherein the hub is coupled to a pusher, wherein the implant is heat set in a first expanded state comprising a dome portion, a brim portion, and an open proximal end; advancing the implant out of the microcatheter into an interior cavity of the cerebral aneurysm by advancing the implant in a distal direction towards a dome of the cerebral aneurysm, wherein the permeable shell expands to a second expanded state in the interior cavity of the aneurysm, wherein the second expanded state comprises an open distal; detaching the pusher from the implant; and withdrawing the microcatheter from the region of interest after detaching the implant.

In some embodiments, the first expanded state has a hat shape.

In some embodiments, the first expanded state has an umbrella shape.

In some embodiments, the second expanded state has a bowl shape.

In some embodiments, the second expanded state further comprises a substantially flat portion at a proximal end.

In some embodiments, the second expanded state has a tulip shape.

In some embodiments, the second expanded state has a cup shape.

In some embodiments, the plurality of elongate filaments are arranged in a braided mesh. In some embodiments, the permeable shell comprises a single layer of the braided mesh. In some embodiments, the permeable shell comprises a double layer of the braided mesh. In some embodiments, the permeable shell comprises multiple layers of the braided mesh.

In some embodiments, the second expanded shape is different than the first expanded shape.

In some embodiments, the hub is located at a distal end of the first expanded state.

In some embodiments, the hub is located at a proximal end of the second expanded state.

In some embodiments, the first end and the second end of each of the plurality of elongate filaments are gathered in a hub.

In some embodiments, each of the plurality of elongate filaments comprises a middle portion, and wherein the middle portion forms a loop at a distal end of the second expanded state.

In some embodiments, each of the plurality of elongate filaments comprises a middle portion, and wherein the middle portion forms a loop at a proximal end of the first expanded state.

In some embodiments, a distal end of the permeable shell in the first expanded state is inverted.

In some embodiments, a distal end of the permeable shell in the first expanded state has an outer convex surface.

In some embodiments, a distal end of the permeable shell in the second expanded state has an inner concave surface.

In many embodiments, a method for treating a cerebral aneurysm having an interior cavity and a neck includes the steps of: advancing an implant in a microcatheter to a region of interest in a cerebral artery, wherein the implant comprises a permeable shell made from a plurality of elongate filaments, wherein each of the plurality of filaments has a first end and a second end, wherein the first end of each of the plurality of elongate filaments are gathered in a hub, wherein the hub is coupled to a pusher; advancing the implant out of the microcatheter into an interior cavity of the cerebral aneurysm by advancing the implant in a distal direction towards a dome of the cerebral aneurysm, wherein the permeable shell expands to a first expanded state in the interior cavity of the aneurysm, wherein the first expanded state comprises a dome portion and a brim portion, wherein the dome portion comprises an outer surface, an inner surface, and an inner cavity defined by the inner surface, and wherein the hub is located in the inner cavity at a distal end of the first expanded state; withdrawing the pusher proximally within the interior cavity, wherein the permeable shell assumes a second expanded state in the interior cavity of the aneurysm, wherein the second expanded state comprises a substantially flat portion at a proximal end and an open distal end, wherein the hub is located at a proximal end of the expanded state; detaching the pusher from the implant; and withdrawing the microcatheter from the region of interest after detaching the implant.

In some embodiments, the first expanded state has a hat shape.

In some embodiments, the first expanded state has an umbrella shape.

In some embodiments, each of the plurality of elongate filaments comprises a middle portion, and wherein the middle portion forms a loop at a distal end of the second expanded state.

In some embodiments, each of the plurality of elongate filaments comprises a middle portion, and wherein the middle portion forms a loop at a proximal end of the first expanded state.

In some embodiments, the plurality of filaments at a distal end of the first expanded state are inverted.

In some embodiments, a height of the permeable shell in the second expanded state is less than a height of the cerebral aneurysm.

In some embodiments, the cerebral aneurysm is a wide-neck cerebral aneurysm.

In some embodiments, the substantially flat portion sits within a neck of the wide-neck cerebral aneurysm after the implant is deployed.

In some embodiments, the hub is detachably coupled to the pusher.

In many embodiments, a device for treatment of a patient's cerebral aneurysm includes a permeable shell made from a plurality of elongate filaments, wherein each of the plurality of filaments has a first end and a second end, wherein the first end of each of the plurality of elongate filaments are gathered in a hub, wherein the permeable shell has a first unrestrained configuration comprising a closed distal end having an outer convex surface, and wherein the permeable shell is configured to assume a second configuration when deployed in the patient's cerebral aneurysm, wherein the second configuration comprises a an open distal end having an inner convex surface.

In some embodiments, the first unrestrained configuration comprises an umbrella shape.

In some embodiments, the hub is not in contact with the outer convex surface.

In some embodiments, the second configuration comprises a bowl shape.

In some embodiments, the hub is not located in the inner cavity of the second configuration.

In many embodiments, a device for treatment of a patient's cerebral aneurysm includes a permeable shell made from a plurality of elongate filaments, wherein each of the plurality of filaments has a first end and a second end, wherein the first end of each of the plurality of elongate filaments are gathered in a hub, wherein the permeable shell has a first unrestrained configuration comprising a closed distal end having an outer convex surface, and wherein the permeable shell is configured to assume a second configuration when deployed in the patient's cerebral aneurysm, wherein the second configuration comprises an open distal end and an inner cavity.

In some embodiments, the first unrestrained configuration comprises an umbrella shape.

In some embodiments, the hub is not in contact with the outer convex surface.

In some embodiments, the second configuration comprises a bowl shape.

In some embodiments, the hub is not located in the inner cavity of the second configuration.

In many embodiments, a device for treatment of a patient's cerebral aneurysm includes a permeable shell made from a plurality of elongate filaments, wherein each of the plurality of filaments has a first end and a second end, wherein the first end of each of the plurality of elongate filaments are gathered in a hub, wherein the permeable shell has a first surface and a second surface, and wherein the hub is attached to the second surface, wherein the permeable shell has a first unrestrained configuration comprising a convex portion having an inner cavity, and wherein the hub is located in the inner cavity of the convex portion in the first unrestrained configuration, wherein the permeable shell is configured to assume a second configuration when deployed in the patient's cerebral aneurysm, wherein the second configuration is an inversion of the first unrestrained configuration.

In some embodiments, a surface of the inner cavity of the convex portion is the second surface.

In some embodiments, the second configuration comprises an open distal end.

In some embodiments, the first unrestrained configuration further comprises an open proximal end.

In some embodiments, the first unrestrained configuration further comprises a planar portion attached to the convex portion. In some embodiments, the planar portion extends from the convex portion at an angle.

### CLAUSES

Exemplary embodiments are set out in the following numbered clauses.

Clause 1. A device for treatment of a patient's cerebral aneurysm, comprising:
a permeable shell made from a plurality of elongate filaments, wherein each of the plurality of filaments has a first end and a second end, wherein the first end of each of the plurality of elongate filaments are gathered in a hub,
wherein the permeable shell has a first unrestrained preset configuration comprising a dome portion and a brim portion, wherein the dome portion comprises an outer surface, an inner surface, and an inner cavity defined by the inner surface, and wherein the hub is located in the inner cavity in the first unrestrained preset configuration, and
wherein the permeable shell is configured to assume a second restrained configuration when deployed in the patient's cerebral aneurysm, wherein the second restrained configuration comprises an open distal end.

Clause 2. The device of clause 1, wherein the first unrestrained preset configuration has a hat shape.

Clause 3. The device of clause 2, wherein the hat shape further comprises a lip portion.

Clause 4. The device of clause 3, wherein the lip portion extends at an acute angle from the brim portion.

Clause 5. The device of clause 1, wherein the first unrestrained preset configuration has an umbrella shape.

Clause 6. The device of clause 1, wherein the hub is located at a distal end of the first unrestrained preset configuration.

Clause 7. The device of clause 1, wherein the second restrained configuration has a bowl shape.

Clause 8. The device of clause 1, wherein the second constrained configuration further comprises a substantially flat portion at a proximal end.

Clause 9. The device of clause 1, wherein the second restrained configuration has a tulip shape.

Clause 10. The device of clause 1, wherein the second restrained configuration has a cup shape.

Clause 11. The device of clause 1, wherein the hub is located at a proximal end of the second restrained configuration.

Clause 12. The device of clause 1, wherein the first end and the second end of each of the plurality of elongate filaments are gathered in a hub.

Clause 13. The device of clause 1, wherein each of the plurality of elongate filaments comprises a middle portion, and wherein the middle portion forms a loop at a distal end of the second restrained configuration.

Clause 14. The device of clause 1, wherein each of the plurality of elongate filaments comprises a middle portion, and wherein the middle portion forms a loop at a proximal end of the first unrestrained preset configuration.

Clause 15. The device of clause 1, wherein a distal end of the permeable shell in the first unrestrained preset configuration is inverted.

Clause 16. The device of clause 1, wherein a distal end of the permeable shell in the first unrestrained preset configuration has an outer convex surface.

Clause 17. The device of clause 1, wherein a distal end of the permeable shell in the second restrained configuration has an inner concave surface.

Clause 18. The device of clause 1, wherein the plurality of elongate filaments are arranged in a braided mesh.

Clause 19. The device of clause 18, wherein the permeable shell comprises a single layer of the braided mesh.

Clause 20. The device of clause 18, wherein the permeable shell comprises a double layer of the braided mesh.

Clause 21. The device of clause 18, wherein the permeable shell comprises multiple layers of the braided mesh.

Clause 22. A method for treating a cerebral aneurysm having an interior cavity and a neck, comprising the steps of:
advancing an implant in a microcatheter to a region of interest in a cerebral artery, wherein the implant comprises a permeable shell made from a plurality of elongate filaments, wherein each of the plurality of filaments has a first end and a second end, wherein the first end of each of the plurality of elongate filaments are gathered in a hub, wherein the hub is coupled to a pusher, wherein the implant is heat set in a first expanded state comprising a dome portion, a brim portion, and an open proximal end;
advancing the implant out of the microcatheter into an interior cavity of the cerebral aneurysm by advancing the implant in a distal direction towards a dome of the cerebral aneurysm, wherein the permeable shell expands to a second expanded state in the interior cavity of the aneurysm, wherein the second expanded state comprises an open distal;
detaching the pusher from the implant; and
withdrawing the microcatheter from the region of interest after detaching the implant.

Clause 23. The method of clause 22, wherein the first expanded state has a hat shape.

Clause 24. The method of clause 22, wherein the first expanded state has an umbrella shape.

Clause 25. The method of clause 22, wherein the second expanded state has a bowl shape.

Clause 26. The method of clause 22, wherein the second expanded state further comprises a substantially flat portion at a proximal end.

Clause 27. The method of clause 22, wherein the second expanded state has a tulip shape.

Clause 28. The method of clause 22, wherein the second expanded state has a cup shape.

Clause 29. The method of clause 22, wherein the plurality of elongate filaments are arranged in a braided mesh.

Clause 30. The method of clause 29, wherein the permeable shell comprises a single layer of the braided mesh.

Clause 31. The method of clause 29, wherein the permeable shell comprises a double layer of the braided mesh.

Clause 32. The method of clause 29, wherein the permeable shell comprises multiple layers of the braided mesh.

Clause 33. The method of clause 22, wherein the second expanded shape is different than the first expanded shape.

Clause 34. The method of clause 22, wherein the hub is located at a distal end of the first expanded state.

Clause 35. The method of clause 22, wherein the hub is located at a proximal end of the second expanded state.

Clause 36. The method of clause 22, wherein the first end and the second end of each of the plurality of elongate filaments are gathered in a hub.

Clause 37. The method of clause 22, wherein each of the plurality of elongate filaments comprises a middle portion, and wherein the middle portion forms a loop at a distal end of the second expanded state.

Clause 38. The method of clause 22, wherein each of the plurality of elongate filaments comprises a middle portion, and wherein the middle portion forms a loop at a proximal end of the first expanded state.

Clause 39. The method of clause 22, wherein a distal end of the permeable shell in the first expanded state is inverted.

Clause 40. The method of clause 22, wherein a distal end of the permeable shell in the first expanded state has an outer convex surface.

Clause 41. The method of clause 22, wherein a distal end of the permeable shell in the second expanded state has an inner concave surface.

Clause 42. A method for treating a cerebral aneurysm having an interior cavity and a neck, comprising the steps of:
advancing an implant in a microcatheter to a region of interest in a cerebral artery, wherein the implant comprises a permeable shell made from a plurality of elongate filaments, wherein each of the plurality of filaments has a first end and a second end, wherein the first end of each of the plurality of elongate filaments are gathered in a hub, wherein the hub is coupled to a pusher;
advancing the implant out of the microcatheter into an interior cavity of the cerebral aneurysm by advancing the implant in a distal direction towards a dome of the cerebral aneurysm, wherein the permeable shell expands to a first expanded state in the interior cavity of the aneurysm, wherein the first expanded state comprises a dome portion and a brim portion, wherein the dome portion comprises an outer surface, an inner surface, and an inner cavity defined by the inner surface, and wherein the hub is located in the inner cavity at a distal end of the first expanded state;
withdrawing the pusher proximally within the interior cavity, wherein the permeable shell assumes a second expanded state in the interior cavity of the aneurysm, wherein the second expanded state comprises a substantially flat portion at a proximal end and an open distal end, wherein the hub is located at a proximal end of the expanded state;
detaching the pusher from the implant; and
withdrawing the microcatheter from the region of interest after detaching the implant.

Clause 43. The method of clause 42, wherein the first expanded state has a hat shape.

Clause 44. The method of clause 42, wherein the first expanded state has an umbrella shape.

Clause 45. The method of clause 42, wherein each of the plurality of elongate filaments comprises a middle portion, and wherein the middle portion forms a loop at a distal end of the second expanded state.

Clause 46. The method of clause 42, wherein each of the plurality of elongate filaments comprises a middle portion, and wherein the middle portion forms a loop at a proximal end of the first expanded state.

Clause 47. The method of clause 42, wherein the plurality of filaments at a distal end of the first expanded state are inverted.

Clause 48. The method of clause 42, wherein a height of the permeable shell in the second expanded state is less than a height of the cerebral aneurysm.

Clause 49. The method of clause 42, wherein the cerebral aneurysm is a wide-neck cerebral aneurysm.

Clause 50. The method of clause 42, wherein the substantially flat portion sits within a neck of the wide-neck cerebral aneurysm after the implant is deployed.

Clause 51. The method of clause 42, wherein the hub is detachably coupled to the pusher.

Clause 52. A device for treatment of a patient's cerebral aneurysm, comprising:
a permeable shell made from a plurality of elongate filaments, wherein each of the plurality of filaments has a first end and a second end, wherein the first end of each of the plurality of elongate filaments are gathered in a hub,
wherein the permeable shell has a first unrestrained configuration comprising a closed distal end having an outer convex surface, and
wherein the permeable shell is configured to assume a second configuration when deployed in the patient's cerebral aneurysm, wherein the second configuration comprises a an open distal end having an inner convex surface.

Clause 53. A device for treatment of a patient's cerebral aneurysm, comprising:
a permeable shell made from a plurality of elongate filaments, wherein each of the plurality of filaments has a first end and a second end, wherein the first end of each of the plurality of elongate filaments are gathered in a hub,
wherein the permeable shell has a first unrestrained configuration comprising a closed distal end having an outer convex surface, and
wherein the permeable shell is configured to assume a second configuration when deployed in the patient's cerebral aneurysm, wherein the second configuration comprises an open distal end and an inner cavity.

Clause 54. The device of clause 53, wherein the first unrestrained configuration comprises an umbrella shape.

Clause 55. The device of clause 53, wherein the hub is not in contact with the outer convex surface.

Clause 56. The device of clause 53, wherein the second configuration comprises a bowl shape.

Clause 57. The device of clause 53, wherein the hub is not located in the inner cavity of the second configuration.

Clause 58. A device for treatment of a patient's cerebral aneurysm, comprising:
a permeable shell made from a plurality of elongate filaments, wherein each of the plurality of filaments has a first end and a second end, wherein the first end of each of the plurality of elongate filaments are gathered in a hub, wherein the permeable shell has a first surface and a second surface, and wherein the hub is attached to the second surface,
wherein the permeable shell has a first unrestrained configuration comprising a convex portion having an inner cavity, and wherein the hub is located in the inner cavity of the convex portion in the first unrestrained configuration,
wherein the permeable shell is configured to assume a second configuration when deployed in the patient's cerebral aneurysm, wherein the second configuration is an inversion of the first unrestrained configuration.

Clause 59. The device of clause 58, wherein a surface of the inner cavity of the convex portion is the second surface.

Clause 60. The device of clause 58, wherein the second configuration comprises an open distal end.

Clause 61. The device of clause 58, wherein the first unrestrained configuration further comprises an open proximal end.

Clause 62. The device of clause 58, wherein the first unrestrained configuration further comprises a planar portion attached to the convex portion.

Clause 63. The device of clause 62, wherein the planar portion extends from the convex portion at an angle.

1. A device for treatment of a patient's cerebral aneurysm, comprising:
a permeable shell made from a plurality of elongate filaments, wherein each of the plurality of filaments has a first end and a second end, wherein the first end of each of the plurality of elongate filaments are gathered in a hub,
wherein the permeable shell has a first unrestrained preset configuration comprising a dome portion and a brim portion, wherein the dome portion comprises an outer surface, an inner surface, and an inner cavity defined by the inner surface, and wherein the hub is located in the inner cavity in the first unrestrained preset configuration, and
wherein the permeable shell is configured to assume a second restrained configuration when deployed in the patient's cerebral aneurysm, wherein the second restrained configuration comprises an open distal end.

2. The device of claim Error! Reference source not found., wherein the first unrestrained preset configuration has a hat shape.

3. The device of claim 2, wherein the hat shape further comprises a lip portion.

4. The device of claim 3, wherein the lip portion extends at an acute angle from the brim portion.

5. The device of claim Error! Reference source not found., wherein the first unrestrained preset configuration has an umbrella shape.

6. The device of claim Error! Reference source not found., wherein the hub is located at a distal end of the first unrestrained preset configuration.

7. The device of claim Error! Reference source not found., wherein the second restrained configuration has a bowl shape.

8. The device of claim Error! Reference source not found., wherein the second constrained configuration further comprises a substantially flat portion at a proximal end.

9. The device of claim Error! Reference source not found., wherein the second restrained configuration has a tulip shape.

10. The device of claim Error! Reference source not found., wherein the second restrained configuration has a cup shape.

11. The device of claim Error! Reference source not found., wherein the hub is located at a proximal end of the second restrained configuration.

12. The device of claim Error! Reference source not found., wherein the first end and the second end of each of the plurality of elongate filaments are gathered in a hub.

13. The device of claim Error! Reference source not found., wherein each of the plurality of elongate filaments comprises a middle portion, and wherein the middle portion forms a loop at a distal end of the second restrained configuration.

14. The device of claim Error! Reference source not found., wherein each of the plurality of elongate filaments comprises a middle portion, and wherein the middle portion forms a loop at a proximal end of the first unrestrained preset configuration.

15. The device of claim Error! Reference source not found., wherein a distal end of the permeable shell in the first unrestrained preset configuration is inverted.

16. The device of claim Error! Reference source not found., wherein a distal end of the permeable shell in the first unrestrained preset configuration has an outer convex surface.

17. The device of claim Error! Reference source not found., wherein a distal end of the permeable shell in the second restrained configuration has an inner concave surface.

18. The device of claim Error! Reference source not found., wherein the plurality of elongate filaments are arranged in a braided mesh.

19. The device of claim 18, wherein the permeable shell comprises a single layer of the braided mesh.

20. The device of claim 18, wherein the permeable shell comprises a double layer of the braided mesh.

21. The device of claim 18, wherein the permeable shell comprises multiple layers of the braided mesh.

22-63. (canceled)
